(19) United States
(12) Patent Application Publication
(10) Pub. No.: US 2026/0157766 A1
(43) Pub. Date: Jun. 11, 2026

(54) CONNECTION MEMBER FOR CONNECTING A DEVICE FOR REMOVING BODY FLUID, PARTICULARLY NASAL FLUID TO AN APPARATUS GENERATING SUCTION

(71) Applicant: ILLÉS CSÓK ÉS TÁRSA KFT., Budapest (HU)

(72) Inventor: Anna ILLÉS CSÓK, Budapest (HU)

(73) Assignee: ILLÉS CSÓK ÉS TÁRSA KFT., Budapest (HU)

(21) Appl. No.: 19/150,137

(22) PCT Filed: Jan. 18, 2024
(86) PCT No.: PCT/HU2024/050003

§ 371 Date: Jul. 22, 2025

(30) Foreign Application Priority Data
Jan. 31, 2023 (HU) P2300044

(51) Int. Cl. A61B 17/24; A61B 17/00

(52) U.S. Cl. CPC A61B 17/24; A61B 2017/00477; A61B 2017/246; A61B 2217/005

(57) ABSTRACT

The invention relates to a connection member (1) for connecting a device for removing exudate, particularly nasal exudate, to an apparatus generating suction, preferably to a vacuum cleaner, comprising a conical skirt (2) that is adapted to be plugged into the suction hose of the vacuum cleaner, and has a frustoconical shape tapering towards the suction hose, an interior portion (4) that is located inside the skirt (2), extending in the direction of the suction hose from the base plate (3) terminating the skirt (2), conically tapering away from the base plate (3), and terminating in a stem (5). The connection member according to the invention is characterised in that at least one and preferably 2-3 opening(s) (6, 7, 8) is (are) formed in the base plate, in an annular base portion (9) thereof around the interior portion (4), and/or in the skirt (2) in the vicinity of the base plate (3).

### TECHNICAL FIELD

The prior art includes various devices for removing unwanted exudates from the human body. An example of known devices for removing, by suction, nasal fluid is a pipette terminated by a rubber cap, wherein the fluid is removed applying vacuum produced by manually compressing the rubber cap. The document with publication number WO99/53819 discloses a solution for removing nasal fluid applying suction generated by a balloon or suction applied by the user. A common drawback of these devices is that they are not capable of completely removing the exudate, and at the same time are either not suited for reuse or reuse involves the risk of infection.

### BACKGROUND ART

For the thorough removal of the exudate, therefore, suction must be increased applying some type of vacuum source.

The appropriate vacuum effect can be provided simply by utilising a vacuum cleaner.

Such a device, i.e., a device that is adapted to be connected to a vacuum cleaner and comprises a collector vessel adapted to collect the removed exudate, is disclosed in the patent description HU 215563.

Numerous problems have arisen in relation to the use of this device, and various technical solutions addressing these problems are included in the prior art.

Such technical solutions are disclosed in the utility models No. U 3471 and U 3472, wherein the suction head of the device is connected to the collector vessel, and the device itself consists of releasably interconnected bottom and upper portions that have conical and arcuate configuration.

A common disadvantage of known devices is that the collector vessel of the device is in many cases incapable of retaining the removed bodily fluid, resulting in that the body fluid enters the interconnection hose of the vacuum cleaner (or the vacuum cleaner itself) due the suction effect resulting from the applied vacuum, causing undesirable contamination.

This is dependent on the consistency of the body fluid being removed.

In recent years, pharmaceutical companies have introduced a number of allegedly antibacterial/medicinal nasal sprays that are capable of dislodging and diluting deposited body fluid.

After dilution, the bodily exudate has a water-like consistency, and therefore it cannot be safely removed applying known devices because the vacuum-generated airflow can carry it off from the collector vessel, transporting it to the vacuum source, i.e., the vacuum cleaner.

This problem is solved by the technical solution according to the patent HU 231 151.

Devices according to the known technical solutions are connected to the apparatus generating suction by means of a connection member insertable in a vacuum cleaner suction hose.

The connection member preferably consists of a frustoconical skirt tapering towards the suction hose that is terminated by a base plate, and an interior portion that is disposed inside the skirt, protruding from the base plate, and tapering in the opposite direction than the frustoconical skirt. The flexible hose of the body fluid removing device is connected to the free part of the interior portion extending over the skirt.

The conventional connection member is a device made integrally by injection moulding, i.e., it is preferably a transparent, rigid plastic device. Such a device is disclosed in the community design No. 000842877.

With modern vacuum cleaners it is not feasible to control “false” airflow, as a result of which the connection member inserted into the suction hose contracts the cross section of the flow path under suction, and also partially blocks it. This causes the control system of the vacuum cleaner to halt the operation of the machine, as a result of which it becomes unable to operate the nasal suction device connected to it.

Another problem is that some suction hoses have non-circular configuration, which makes them unable to receive the connection member.

### DISCLOSURE OF INVENTION

The objective of the present invention is to eliminate the above-described problems and provide a connection member suited for connecting nasal suction devices.

The configuration of the connection member according to the invention is based on the recognition that the connection can at the same time also be applied for controlling the “false” airflow occurring in relation to the suction effect.

The objective of the invention is fulfilled by providing a connection member that comprises a conical skirt that is adapted to be plugged into the suction hose of the vacuum cleaner, and has a frustoconical shape tapering towards the suction hose, an interior portion that is located inside the skirt, extending in the direction of the suction hose from a base plate terminating the skirt, conically tapering away from the base plate, and terminating in a stem, and that is characterised in that at least one and preferably 2-3 opening(s) is (are) formed in the base plate, on an annular base portion thereof around the interior portion, and/or in the skirt in the vicinity of the base plate.

In a preferred embodiment of the connection member according to the invention, the opening(s) of the annular base portion of the base plate has (have) circular shape, wherein the diameter of the opening(s) of the base plate is smaller than the distance between the skirt and the interior portion.

In a preferred embodiment of the connection member according to the invention, the opening(s) of the skirt has(have) quadrangular shape.

In a further preferred embodiment of the connection member according to the invention, the opening located in the skirt is formed penetrating into the base plate.

All preferred embodiments of the connection member according to the invention are made of resilient plastic material.

### BRIEF DESCRIPTION OF DRAWINGS

A preferred embodiment of the connection member according to the invention will be described in detail below referring to the accompanying drawings, where

FIG. 1 is the side view of a prior art connection member,

FIG. 2 is a view taken along the line A-A of FIG. 1, and

FIGS. 3-5 are perspective views of the connection member according to the invention.

### BEST MODE OF CARRYING OUT THE INVENTION

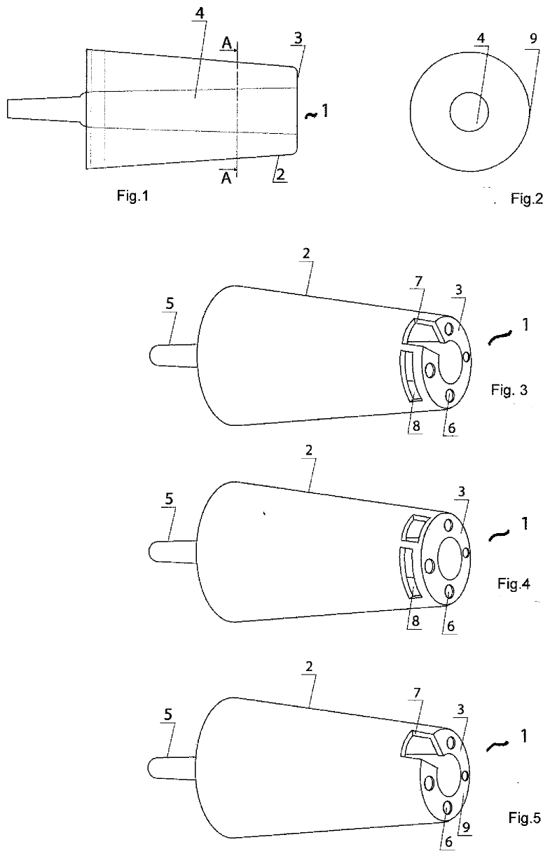

As can be seen in FIG. 1, the prior art connection member 1 is a frustoconical body that can be plugged in a vacuum cleaner suction hose and has a shape tapering in the direction of the suction hose, the body having an interior portion 4 that extends from the base plate 3 terminating the skirt 2 of the frustoconical body and tapers in an opposite direction than the frustoconical shape of the body, terminating in a tapering stem 5 at the end of the skirt 2.

As can be seen in FIG. 2, an annular base portion 9 is located on the base plate 3 of the connection member 1, between the skirt 2 and the interior portion 3.

FIGS. 3-5 illustrate various embodiments of the connection member 1 according to the invention in perspective view.

The configuration of the connection member 1 is essentially identical to the configuration of the known technical solution according to FIG. 1, with the significant difference that openings 6, 7 and 8 are formed in the base plate 3 and/or in the skirt 2.

By including a single opening, the “false” airflow can be controlled in the state in which the device is inserted into the suction hose, which enables the operation of the nasal suction device connected to the vacuum cleaner by the connection member 1.

In FIG. 3 such a configuration can be seen wherein four openings 6 are disposed on the annular base portion 9 of the base plate 3, but the number of the openings 6 may vary from this.

In FIG. 3, an opening 8 is also disposed on the skirt 2, said opening being a square opening extending along the skirt 2 in the middle of the base plate 3; furthermore, this embodiment also comprises an opening 7 that penetrates into the annular base portion 9 of the base plate 3, i.e., in the case of this embodiment the portion between the two openings 6 of the annular base portion 9 is cut out.

In FIG. 4, the openings 6 are formed in the annular base portion 9 of the base plate 3, and also in the skirt 2, in the vicinity of the base plate 3.

In FIG. 5 such an embodiment is illustrated in which the connection member 1 is provided with openings 6 on the annular base portion 9 of the base plate 3, while the opening 7 in the skirt 2 penetrates into the annular base portion 9 in between the two openings 6 of the base plate 3.

The connection member 1 is preferably made integrally, of a resilient plastic material.

Thanks to its resilient material, the skirt is able to connect the nasal suction device even to vacuum cleaners having a suction hose with a non-circular end.

Furthermore, it also ensures the connectivity of the nasal suction device in the case of vacuum cleaners with hoses that have ribs or other features constricting the inside space of the hose.

### LIST OF REFERENCE NUMERALS

1 connection member
2 skirt
3 base plate
4 interior portion
5 stem
6 opening
7 opening
8 opening
9 annular base portion

1. A connection member (1) for connecting a device for removing exudate, particularly nasal exudate, to a device generating suction, preferably to a vacuum cleaner, comprising a conical skirt (2) that is adapted to be plugged into a suction hose of the device generating suction, and has a frustoconical shape tapering towards the suction hose, an interior portion (4) defined inside the skirt (2), extending in the direction of the suction hose from a base plate (3) terminating the skirt (2), conically tapering away from the base plate (3), and terminating in a stem (5), characterised in that at least one opening (6) is present in the base plate, in an annular base portion (9) thereof.

2. The connection member (1) according to claim 1, characterised in that the opening (6) has a circular shape.

3. The connection member (1) according to claim 1, characterised in that an additional opening is defined in the skirt (2) and has a quadrangular shape.

4. The connection member (1) according to claim 1, characterised in that an additional opening (7) is provided in the skirt (2) and extends into the base plate (3).

5. The connection member (1) according to claim 1 characterised in that the opening (6) in the base plate (3) is smaller than the distance between the skirt (2) and the interior portion (4).

6. The connection member (1) according claim 1 made of a resilient plastic material.

7. The connection member (1) according claim 1 also having an opening (8) in the skirt and an additional opening (7) defined by the skirt and the base plate (3).

8. A connection member for connecting a device for removing exudate to a device generating suction which comprises a hollow frustoconical housing defined by a tapered skirt, an annular base plate at a first end thereof, and a stem provided with a through passageway at a second end thereof; the first end being adapted to be received into a suction hose of the device generating suction and the annular base plate defining at least one opening in the annular portion thereof.
